(19) United States
(12) Patent Application Publication
(10) Pub. No.: US 2022/0022913 A1
(43) Pub. Date: Jan. 27, 2022

(54) PUNCTURE-TREATMENT ASSEMBLY

(71) Applicant: Baylis Medical Company Inc., Montreal (CA)

(72) Inventor: Moussa Chehade, Toronto (CA)

(21) Appl. No.: 17/382,938

(22) Filed: Jul. 22, 2021

Related U.S. Application Data
(60) Provisional application No. 63/056,089, filed on Jul. 24, 2020.

(51) Int. Cl. A61B 17/34

(52) U.S. Cl. CPC A61B 17/3417; A61B 17/3496

(57) ABSTRACT

The elongated introducer assembly is configured to be selectively movable proximate to the biological feature of the patient, and puncture the biological feature of the patient and form a puncture site through the biological feature; and dilate the puncture site; and receive the auxiliary device for facilitation of movement of the auxiliary device toward the puncture site.

### TECHNICAL FIELD

This document relates to the technical field of (and is not limited to) (A) an elongated introducer assembly that is movable proximate to a biological feature, and has a puncture-treatment assembly (and method thereof), and/or (B) a puncture-treatment assembly (also called a puncture-management assembly) that is movable proximate to a biological feature (and method thereof).

### BACKGROUND

Known medical devices are configured to facilitate a medical procedure, and help healthcare providers diagnose and/or treat medical conditions of sick patients.

### SUMMARY

It will be appreciated that there exists a need to mitigate (at least in part) at least one problem associated with the existing (known) tissue puncturing devices. After much study of, and experimentation with, the existing (known) tissue puncturing devices, an understanding (at least in part) of the problem and its solution have been identified (at least in part) and are articulated (at least in part) as follows:

For any medical procedure where devices are inserted into the patient's circulatory system, there is a risk for the chance that air and/or foreign material may become mobile (embolism) along the patient's circulatory system for the case where the device is inserted into and/or withdrawn from the patient. One such procedure may be the transseptal access. In current medical practice, a guidewire device, a sheath device, a dilator device and a puncture device (in synergistic cooperation) are each separately insertable into the patient (for performing various puncture management tasks, such as movement of the puncture device for puncture formation, movement of the dilation device for dilation of the puncture site after the formation thereof, etc.).

It may be desirable to reduce the risk of an embolism and/or to reduce the length of procedural time, by reducing, at least in part, the number of devices to be inserted into the patient and used in known procedures.

To mitigate, at least in part, at least one problem associated with the existing technology, there is provided (in accordance with a major aspect) an apparatus. The apparatus is for use on a biological feature of a patient. The apparatus includes and is not limited to (comprises) an elongated introducer assembly having a longitudinal axis extending therethrough, and including a distal portion configured to be selectively movable proximate to the biological feature of the patient. The distal portion, of the elongated introducer assembly, includes a puncture-treatment assembly configured to puncture the biological feature of the patient, and form a puncture site through the biological feature (in response to selective movement of the distal portion toward the biological feature). The distal portion is configured to dilate the puncture site (in response to further selective movement of the distal portion through the puncture site after the puncture site is formed by the puncture-treatment assembly). The elongated introducer assembly and the distal portion (in combination, preferably) define an elongated introducer lumen. The introducer lumen is configured to receive an auxiliary device for facilitation of movement of the auxiliary device through the puncture site extending through the biological feature.

To mitigate, at least in part, at least one problem associated with the existing technology, there is provided (in accordance with a major aspect) an apparatus. The apparatus is for use on a biological feature of a patient. The apparatus includes and is not limited to (comprises) an elongated introducer assembly configured to: (A) be selectively movable proximate to the biological feature of the patient; (B) to puncture the biological feature of the patient; (C) to form a puncture site through the biological feature; (D) dilate the puncture site; and (E) receive the auxiliary device for facilitation of movement of the auxiliary device toward the puncture site.

To mitigate, at least in part, at least one problem associated with the existing technology, there is provided (in accordance with a major aspect) a method. The method is for positioning an auxiliary device proximate to a biological feature of a patient. The method includes and is not limited to (comprises) selectively moving an elongated introducer assembly, having a longitudinal axis extending therethrough, and including a distal portion, proximate to the biological feature of the patient, in which the elongated introducer assembly and the distal portion define an elongated introducer lumen. The method also includes puncturing the biological feature of the patient and forming a puncture site through the biological feature with a puncture-treatment assembly of the distal portion by selectively moving the distal portion toward the biological feature. The method also includes dilating the puncture site with the distal portion by further selective movement of the distal portion through the puncture site after the puncture site is formed by the puncture-treatment assembly. The method also includes facilitating movement of the auxiliary device along the elongated introducer lumen and toward the puncture site.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1, FIG. 2, FIG. 3 and FIG. 4 depict perspective views of embodiments (implementations) of an elongated introducer assembly including a puncture-treatment assembly; and

FIG. 5, FIG. 6, FIG. 7 and FIG. 8 depict perspective views of embodiments (implementations) of the elongated introducer assembly and the puncture-treatment assembly of FIG. 1.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### LISTING OF REFERENCE NUMERALS USED IN THE DRAWINGS

introducer assembly 200
longitudinal axis 201
distal portion 202
puncture-treatment assembly 203
movable spines (204A, 204B, 204C)
puncturing-tapered arrangement 206
dilation-tapered arrangement 208
pull device 210
biological feature 900
patient 901
puncture site 902

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

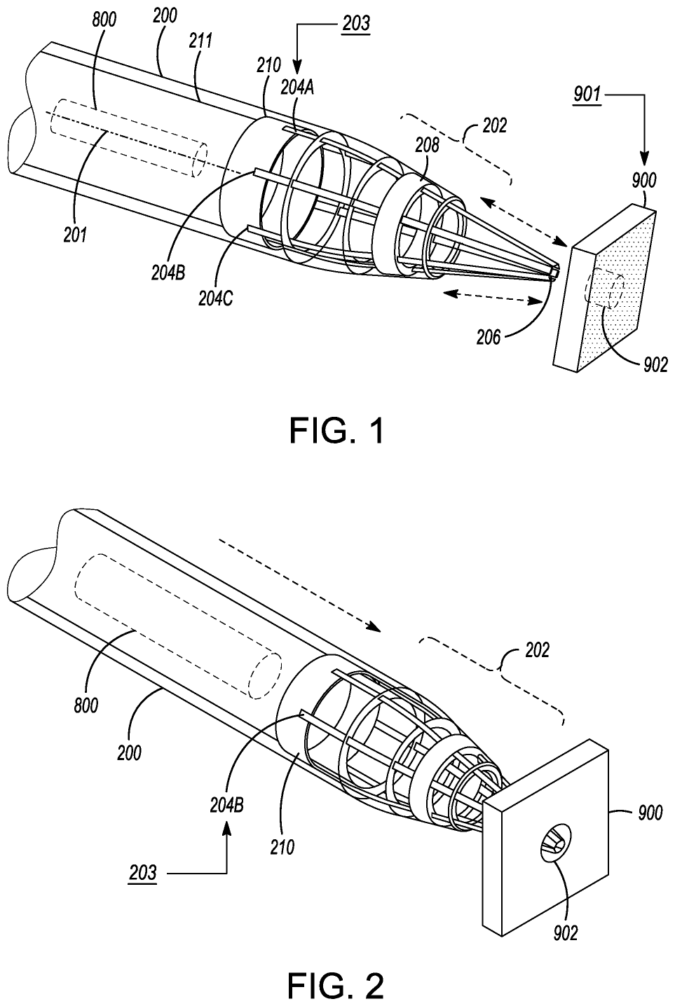

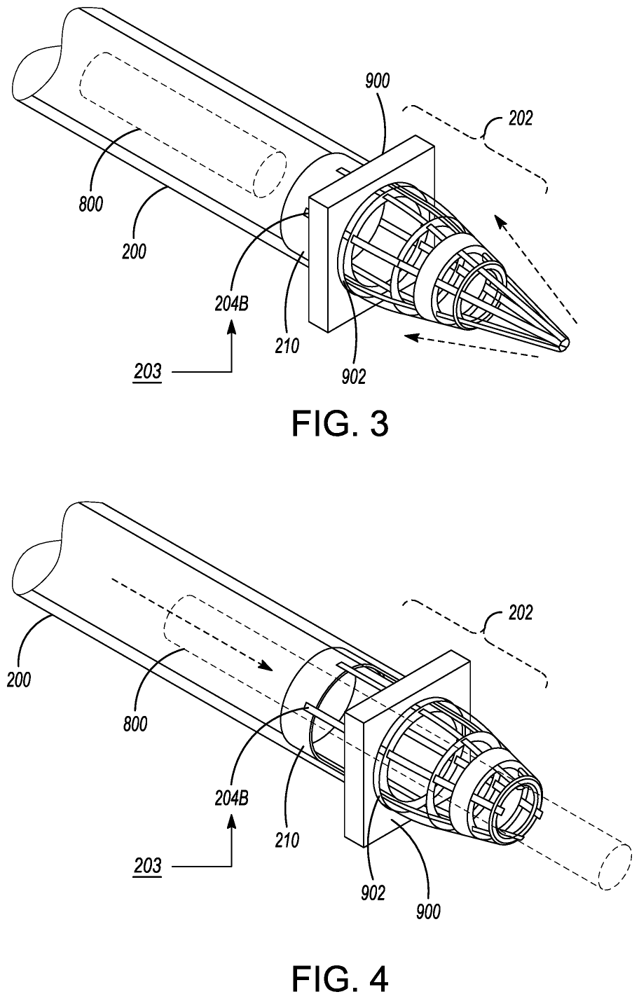

FIG. 1, FIG. 2, FIG. 3 and FIG. 4 depict perspective views of embodiments (implementations) of an elongated introducer assembly 200 including a puncture-treatment assembly 203 (in accordance with a first embodiment).

Referring to the embodiments (implementations) as depicted in FIG. 1 and FIG. 2, the elongated introducer assembly 200 has a longitudinal axis 201 extending therethrough. The elongated introducer assembly 200 includes a distal portion 202. The distal portion 202 is configured to be selectively movable proximate to a biological feature 900 of a patient 901. The distal portion 202 (of the elongated introducer assembly 200) includes the puncture-treatment assembly 203. The puncture-treatment assembly 203 is mounted (operatively mounted) to the distal portion 202. The puncture-treatment assembly 203 is, preferably, configured to be deployed coaxially along the longitudinal axis 201. The puncture-treatment assembly 203 is configured to puncture the biological feature 900 of the patient 901 and (thereby) form a puncture site 902 through the biological feature 900; this is done, preferably, in response to selective movement of the distal portion 202 toward the biological feature 900. The distal portion 202 is configured to dilate the puncture site 902; this is done, preferably, in response to further selective movement of the distal portion 202 through the puncture site 902 (that is, after the puncture site 902 is formed by the puncture-treatment assembly 203). The elongated introducer assembly 200 and the distal portion 202 (in combination, preferably) define an elongated introducer lumen 211 (extending, at least in part, along the longitudinal axis 201). The elongated introducer lumen 211 is configured to receive an auxiliary device 800 for facilitation of movement of the auxiliary device 800 through the puncture site 902 extending through the biological feature 900 (which is also depicted in FIG. 9 and FIG. 10). The puncture-treatment assembly 203 may be called a puncture-management assembly, etc., and any equivalent thereof.

Referring to the embodiment (implementation) as depicted in FIG. 1, the elongated introducer assembly 200 is configured to: (A) be selectively movable proximate to the biological feature 900 of the patient 901; (B) puncture the biological feature 900 of the patient 901 and form a puncture site 902 through the biological feature 900; (C) dilate the puncture site 902; and (D) receive the auxiliary device 800 for facilitation of movement of the auxiliary device 800 toward the puncture site 902.

Referring to the embodiment (implementation) as depicted in FIG. 1, the elongated introducer assembly 200 is configured to be positioned (maneuvered) proximate to the biological feature 900. The elongated introducer assembly 200 may include, for instance, an elongated introducer sheath, an access sheath, etc., and/or any equivalent thereof. The elongated introducer assembly 200 is configured to be inserted into a confined space (or tortuous space) defined by a living body (of the patient). The elongated introducer assembly 200 is (preferably) impermeable by a bodily fluid located in the confined space defined by the living body.

Referring to the embodiment (implementation) as depicted in FIG. 1 and FIG. 2, the puncture-treatment assembly 203 includes (preferably, and is not limited to) movable spines (204A, 204B, 204C) and/or any equivalent thereof. The puncture-treatment assembly 203, or the movable spines (204A, 204B, 204C), may include, for instance, nitinol and/or SAE (the Society of Automotive Engineering) Type 304 Stainless Steel. SAE Type 304 stainless steel contains both chromium (from between about 15% to about 20%) and nickel (from between about 2% to about 10.5%) metals as the main non-iron constituents. The movable spines (204A, 204B, 204C) may include elongated spines, elongated prongs, protrusions and/or any equivalent thereof. The movable spines (204A, 204B, 204C) may include a metallic alloy, such as a memory-shape alloy, etc., and any equivalent thereof. The movable spines (204A, 204B, 204C) are configured to be selectively deployable (movable, maneuverable) between: (A) a puncturing and dilation orientation (as depicted in FIG. 1 or FIG. 2 in which the movable spines (204A, 204B, 204C) are moved to be extended, at least in part, from the interior of the distal portion 202), and (B) an introduction orientation (as depicted in FIG. 4 in which the movable spines (204A, 204B, 204C) are moved to be retracted, at least in part, into the distal portion 202). It will be appreciated that the puncturing and dilation orientation is depicted in FIG. 2 and FIG. 3, and the access and introduction orientation is depicted in FIG. 4. The movable spines (204A, 204B, 204C) are configured to be selectively deployable between the puncturing orientation (as depicted in FIG. 2) and the dilation orientation (as depicted in FIG. 4) relative to the biological feature 900 (preferably, after the distal portion 202, in use, is moved proximate to the biological feature 900, and after the puncture-treatment assembly 203 is moved proximate to the biological feature 900). The movable spines (204A, 204B, 204C) are configured to be selectively deployable (movable, maneuverable) from the elongated introducer assembly 200 (such as from the distal portion 202 of the elongated introducer assembly 200). The movable spines (204A, 204B, 204C) are configured to be mounted, and positioned, at a position located proximate to (or at) the distal portion 202 of the elongated introducer assembly 200. It will be appreciated that the elongated introducer assembly 200 may include respective dedicated lumens (not depicted) configured to respectively receive each of the movable spines (204A, 204B, 204C), if so desired.

Referring to the embodiment (implementation) as depicted in FIG. 1, the movable spines (204A, 204B, 204C) are configured to be positioned in a spaced-apart circumferential relationship around the outer circumference of the distal portion 202.

Referring to the embodiment (implementation) as depicted in FIG. 1, the puncture-treatment assembly 203, or the movable spines (204A, 204B, 204C), is/are configured to puncture the biological feature 900. In accordance with a first option, the puncture-treatment assembly 203, or the movable spines (204A, 204B, 204C), is/are configured to conduct (convey or deliver) energy (such as radiofrequency energy) for puncturing the biological feature 900 (for the formation of the puncture site 902). In accordance with a second option, the puncture-treatment assembly 203, or the movable spines (204A, 204B, 204C), provide(s) at least one mechanically sharp tip configured to puncture the biological feature 900 (for the formation of the puncture site 902). For instance, the puncture-treatment assembly 203, or the movable spines (204A, 204B, 204C), may include (and is/are not limited to) a radiofrequency puncture device, such as the BAYLIS (TRADEMARK) POWERWIRE (REGISTERED TRADEMARK) radio frequency guidewire manufactured by BAYLIS MEDICAL COMPANY (headquartered in Canada). For instance, the puncture-treatment assembly 203, or the movable spines (204A, 204B, 204C), may include (and is/are not limited to) at least one (or more) mechanical cutting portions.

Referring to the embodiment (implementation) as depicted in FIG. 1, the elongated introducer assembly 200 includes, preferably, a pull device 210. The pull device 210 may include (and is not limited to) a ring structure, and/or any equivalent thereof. The pull device 210 is configured to be movable (reciprocally) along the longitudinal axis 201 of the elongated introducer assembly 200. The puncture-treatment assembly 203, or the movable spines (204A, 204B, 204C), is/are fixedly attached to the pull device 210. The distal portion 202 presents a tapered guide (tapered arrangement or geometry) configured to guide movement of the puncture-treatment assembly 203, or the movable spines (204A, 204B, 204C).

Referring to the embodiment (implementation) as depicted in FIG. 1, the puncture-treatment assembly 203 includes (preferably) a shape-memory material configured to be manipulated and/or deformed followed by a return to the original shape that the shape-memory material was set in (prior to manipulation). Shape-memory materials (SMMs) are known and not further described in detail. Shape-memory materials are configured to recover their original shape from a significant and seemingly plastic deformation in response to a particular stimulus applied to the shape-memory material. This is known as the shape memory effect (SME). Superelasticity (in alloys) may be observed once the shape-memory material is deformed under the presence (an application) of a stimulus force.

Referring to the embodiment (implementation) as depicted in FIG. 1, the distal portion 202 forms a dilation-tapered arrangement 208, in which a frontal section of the puncture-treatment assembly 203, or the frontal sections of the movable spines (204A, 204B, 204C), is/are covered (sheltered), at least in part, by the distal portion 202. For instance, the dilation-tapered arrangement 208 includes (preferably) the movable spines (204A, 204B, 204C) and any equivalent thereof. For FIG. 1 to FIG. 4, the dilation-tapered arrangement 208 (or the movable spines (204A, 204B, 204C)) are configured to dilate the puncture site 902 (in response to movement of the dilation-tapered arrangement 208). The dilation-tapered arrangement 208 is configured to dilate, at least in part, the puncture site 902 formed by the puncture-treatment assembly 203 and/or the movable spines (204A, 204B, 204C) (as depicted in FIG. 3). The dilation-tapered arrangement 208 provides (presents) a sloped dilation surface (sloped relative to the longitudinal axis 201 of the elongated introducer assembly 200). The dilation-tapered arrangement 208 is configured to dilate the size of the puncture site 902, up to the size of an outer diameter of the elongated introducer assembly 200 (as depicted in FIG. 3).

Referring to the embodiment (implementation) as depicted in FIG. 1, the puncture-treatment assembly 203, or the movable spines (204A, 204B, 204C), is/are configured to be axially extendable (circumferentially) from (the outer circumference of) the distal portion 202 (that is axially along the longitudinal axis 201). The puncture-treatment assembly 203, or the movable spines (204A, 204B, 204C), is/are configured to be movable, axially relative to the longitudinal axis 201 of the elongated introducer assembly 200. The puncture-treatment assembly 203, or the movable spines (204A, 204B, 204C), is/are also configured to selectively extend into, and retract out of, the elongated introducer assembly 200 (preferably from the distal portion 202 thereof).

Referring to the embodiment (implementation) as depicted in FIG. 1, the puncture-treatment assembly 203 is configured to be selectively extended (advanced) from the interior of the distal portion 202 toward a puncturing-tapered arrangement 206 (as depicted in FIG. 1 or FIG. 2), in preparation for the formation of the puncture site 902 to be formed through the biological feature 900. The puncturing-tapered arrangement 206 is positioned (located) forwardly from (and is spaced apart from) the distal portion 202.

Referring to the embodiment (implementation) as depicted in FIG. 1, the elongated introducer assembly 200 has a longitudinal axis 201 extending therethrough. The elongated introducer assembly 200 includes the distal portion 202 configured to be movable proximate to the biological feature 900 of the patient 901. The distal portion 202, of the elongated introducer assembly 200, includes (preferably) a relatively soft portion or section configured to prevent inadvertent damage to the tissues of the patient's anatomy. The puncture-treatment assembly 203 is selectively deployable from (the distal portion 202 of) the elongated introducer assembly 200. A technical effect (advantage) of the above arrangement is that fewer components (for performing puncturing and dilation tasks) may be utilized. The above arrangement may be utilized (preferably) for transseptal puncture procedures (for the heart of the patient). More generally, the above arrangement may be usable for other cardiovascular procedures where a sheath introducer may need to be passed through a wall of tissue, etc. More generally, the above arrangement may be usable for the case where a tube-like device needs to be passed through a wall of tissue material. More generally, the above arrangement may be usable for the case where the material may be perforated through application of radiofrequency energy, etc.

Referring to the embodiment (implementation) as depicted in FIG. 1, the biological feature 900 (of the patient 901) may include a tissue wall, etc., and any equivalent thereof. The distal portion 202 may be called a distal tip, a sheath tip, etc., and any equivalent thereof. The components of the elongated introducer assembly 200 and the puncture-treatment assembly 203 include, preferably, biocompatible material properties suitable for sufficient performance (such as, dielectric strength, thermal performance, insulation and corrosion, water resistance and/or heat resistance, etc.) for compliance with industrial and/or regulatory safety standards (or compatible for medical usage), etc. Reference is made to the following publication for consideration in the selection of suitable materials: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

Referring to the embodiment (implementation) as depicted in FIG. 2, after the puncturing-tapered arrangement 206 has been formed (by extending the puncture-treatment assembly 203, the movable spines (204A, 204B, 204C) or any equivalent thereof), the puncture-treatment assembly 203 is utilized for formation of the puncture site 902 (such as by activating the emission of energy, such as radiofrequency energy, from the puncture-treatment assembly 203, etc.). For instance, after the puncture-treatment assembly 203 is activated, the distal portion 202 and the puncture-treatment assembly 203 are moved (forwardly) toward the biological feature 900 and the puncture-treatment assembly 203 is used (or remains activated) while the distal portion 202 is moved forwardly for puncture formation (that is, for the formation of the puncture site 902 through the biological feature 900).

Referring to the embodiment (implementation) as depicted in FIG. 3, after the puncture site 902 has been initially formed (in response to movement of the distal portion 202 and the frontal section of the puncture-treatment assembly 203 toward the biological feature 900 while the puncture-treatment assembly 203 remains activated to emit energy), the distal portion 202 and the puncture-treatment assembly 203 are further moved forwardly to thereby impart a dilation force to the puncture site 902 (that is, to increase the dimeter of the puncture site 902); it will be appreciated that the puncture-treatment assembly 203 remains activated while the distal portion 202 is moved forwardly (that is, the puncture-treatment assembly 203 is utilized for the dilation of the puncture site 902 after the puncture site 902 has been initially formed). After the puncture site 902 has been dilated, the puncture-treatment assembly 203 is also configured to be selectively deactivated and then retracted back into the interior of the distal portion 202 (that is, the puncture-treatment assembly 203 is withdrawn, at least in part, back into the interior of the distal portion 202) in preparation for extending the auxiliary device 800 from the interior of the elongated introducer assembly 200 and the distal portion 202 toward (and/or into) the puncture site 902, etc. It will be appreciated that, in accordance with an option, the puncture-treatment assembly 203 may be remain deactivated after the puncture site 902 has been initially formed, and the puncture-treatment assembly 203 may be utilized to passively dilate the puncture site 902 (that is, without the emission of energy from the puncture-treatment assembly 203, if so desired). The action of moving the puncture-treatment assembly 203 through the puncture site 902 while it is in the dilation orientation (as depicted in FIG. 1 and FIG. 2). It will be appreciated that dilation action may occur either with or without activation of the puncture-treatment assembly 203.

Referring to the embodiment (implementation) as depicted in FIG. 4, after the puncture site 902 has been dilated, the puncture-treatment assembly 203 is turned off (deactived to stop emitting energy, etc., if the puncture-treatment assembly 203 was previously activated), and the puncture-treatment assembly 203 is selectively retracted (preferably, into the interior of the distal portion 202 or withdrawn, at least in part, back into the interior of the distal portion 202). Then, the auxiliary device 800 is moved (urged to move) forwardly so that the distal portion of the auxiliary device 800 may become inserted or extended (at least in part) into the puncture site 902 (or moved closer to the biological feature 900), etc.

Referring to the embodiments (implementations) as depicted in FIG. 1, FIG. 2, FIG. 3 and FIG. 4, there is provided a method for positioning the auxiliary device 800 proximate to the biological feature 900 of the patient 901. The method includes (with reference to FIG. 1) selectively moving the elongated introducer assembly 200 (having the longitudinal axis 201 extending therethrough and including the distal portion 202) proximate to the biological feature 900 of the patient 901 (in which the elongated introducer assembly 200 and the distal portion 202 defines the elongated introducer lumen 211 extending therealong). The method also includes (with reference to FIG. 2) puncturing the biological feature 900 of the patient 901 and forming a puncture site 902 through the biological feature 900 with a puncture-treatment assembly 203 of the distal portion 202 by selectively moving the distal portion 202 toward the biological feature 900. The method also includes (with reference to FIG. 3) dilating the puncture site 902 with the distal portion 202 by further selective movement of the distal portion 202 through the puncture site 902 after the puncture site 902 is formed by the puncture-treatment assembly 203. The method also includes (with reference to FIG. 4) facilitating movement of the auxiliary device 800 along the elongated introducer lumen 211 and toward the puncture site 902.

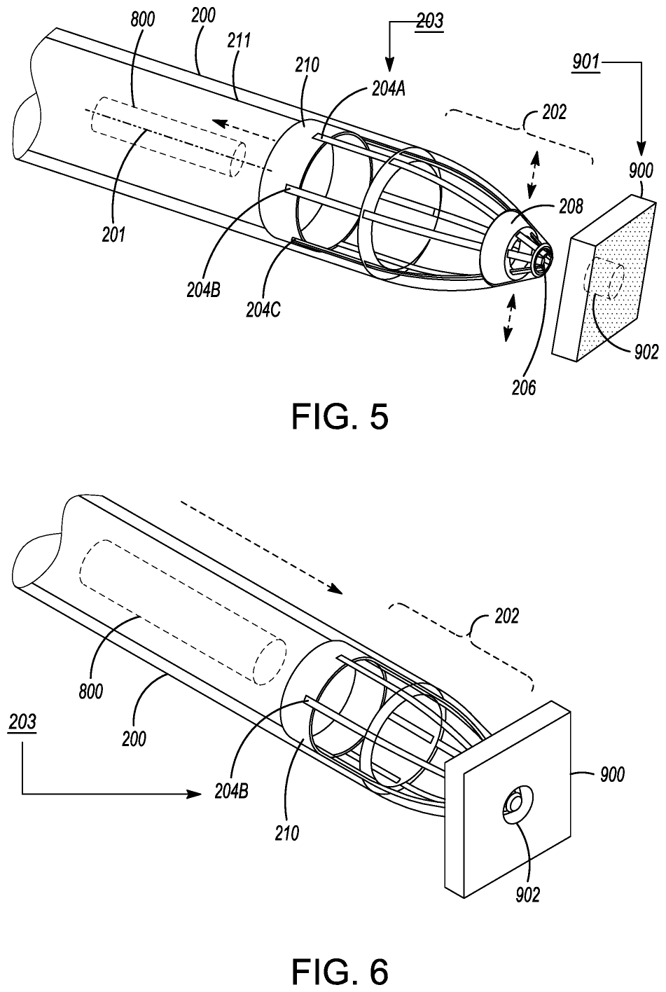

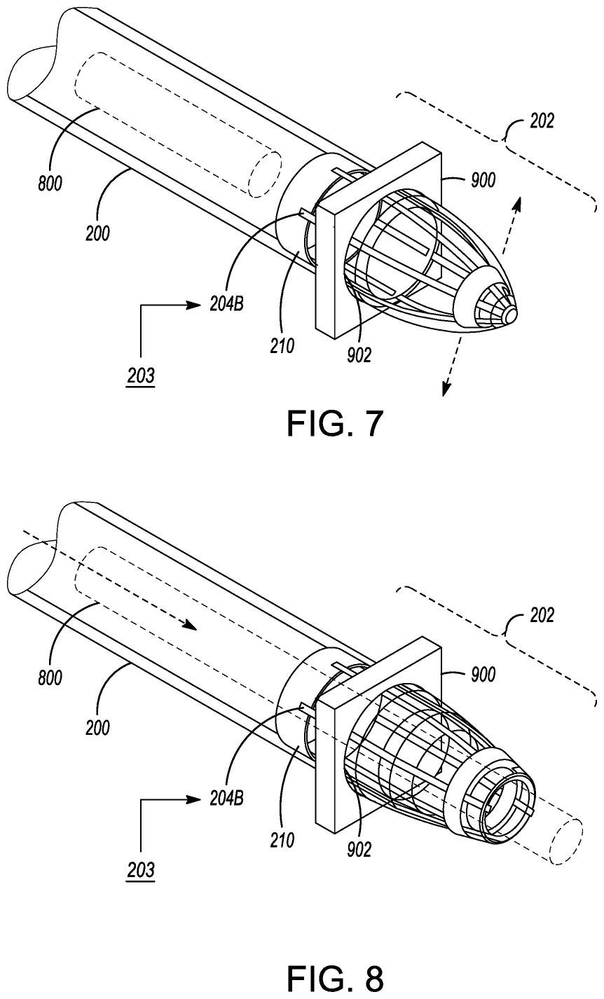

FIG. 5, FIG. 6, FIG. 7 and FIG. 8 depict perspective views of embodiments (implementations) of the elongated introducer assembly 200 and the puncture-treatment assembly 203 of FIG. 1 (in accordance with a second embodiment).

Referring to the embodiment (implementation) as depicted in FIG. 5, the puncture-treatment assembly 203 is configured to be selectively collapsible or foldable toward the puncturing-tapered arrangement 206 (in preparation for, or prior to, the formation of the puncture site 902). The puncture-treatment assembly 203 is also configured to be selectively expanded, as depicted in FIG. 7 (in preparation for dilation of the puncture site 902 after the puncture site 902 is initially formed). It will be appreciated that the expansion may be primarily to allow passage of the auxiliary device 800, and that the distal portion may be more effective collapsed for dilation (if desired).

Referring to the embodiment (implementation) as depicted in FIG. 5, the puncture-treatment assembly 203, or the movable spines (204A, 204B, 204C), is/are configured to be radially extendable (that is, radially extendable, or movable, from the longitudinal axis 201). The puncture-treatment assembly 203, or the movable spines (204A, 204B, 204C), is/are configured to be movable radially from (relative to) the longitudinal axis 201 of the elongated introducer assembly 200. The puncture-treatment assembly 203, or the movable spines (204A, 204B, 204C) remain substantially covered by the distal portion 202. The distal portion 202, of the elongated introducer assembly 200, is configured to be selectively deflected (at least in part) to selectively expand or contract (the outer diameter of) the distal portion 202; this is done, preferably, in response to selective radial movement of the puncture-treatment assembly 203 or the movable spines (204A, 204B, 204C). The puncture-treatment assembly 203, or the movable spines (204A, 204B, 204C), is/are also configured to selectively deflect (to expand and contract) the distal portion 202 of the elongated introducer assembly 200. The distal portion 202 may include a flexible material that permits, at least in part, deflected expansion and contraction of the distal portion 202.

Referring to the embodiment (implementation) as depicted in FIG. 6, after the puncturing-tapered arrangement 206 has been formed (such as, by extending the puncture-treatment assembly 203 or the movable spines (204A, 204B, 204C) or any equivalent thereof), the puncture-treatment assembly 203 is utilized for the formation of the puncture site 902 (such as, by activating the emission of energy, such as radiofrequency energy, from the puncture-treatment assembly 203, etc.). For instance, after the puncture-treatment assembly 203 is activated (utilized), the distal portion 202 and the puncture-treatment assembly 203 are moved (forwardly) toward the biological feature 900 and the puncture-treatment assembly 203 is used (or remains activated) while the distal portion 202 is moved forwardly for puncture formation (that is, for the formation of the puncture site 902 through the biological feature 900).

Referring to the embodiment (implementation) as depicted in FIG. 7, after the puncture site 902 has been initially formed (in response to movement of the distal portion 202 and the frontal section of the puncture-treatment assembly 203 toward the biological feature 900 while the puncture-treatment assembly 203 remains activated), the distal portion 202 and the puncture-treatment assembly 203 are further moved forwardly to thereby impart a dilation force to the puncture site 902 (that is, to increase the dimeter of the puncture site 902); it will be appreciated that the puncture-treatment assembly 203 remains activated while the distal portion 202 is moved forwardly (that is, the puncture-treatment assembly 203 is utilized for the dilation of the puncture site 902 after the puncture site 902 has been initially formed). It will be appreciated that for this embodiment the puncture-treatment assembly 203 may only need to be activated (for emitting energy) while creating the initial puncture site 902 and does not necessary need to be activated during dilation of the puncture hole 902. After the puncture site 902 has been dilated, the puncture-treatment assembly 203 is also configured to be selectively deactivated and then retracted back into the interior of the distal portion 202 (that is, the puncture-treatment assembly 203 is withdrawn, at least in part, back into the interior of the distal portion 202) in preparation for extending the auxiliary device 800 from the interior of the elongated introducer assembly 200 and the distal portion 202 toward, and/or into, the puncture site 902, etc.

Referring to the embodiment (implementation) as depicted in FIG. 8, after the puncture site 902 has been dilated, the puncture-treatment assembly 203 is turned off (that is, de-actived to stop emitting energy, etc. for the case where the puncture-treatment assembly 203 was previously activated), and the puncture-treatment assembly 203 is selectively retracted (preferably, into the interior of the distal portion 202 or withdrawn, at least in part, back into the interior of the distal portion 202). Then, the auxiliary device 800 is moved (urged to move) forwardly so that the distal portion of the auxiliary device 800 may become inserted, or extended, (at least in part) into the puncture site 902 (or moved closer to the biological feature 900), etc.

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

What is claimed is:

1. An apparatus for use with an auxiliary device and a biological feature of a patient, the apparatus comprising:
an elongated introducer assembly configured to be selectively movable proximate to the biological feature of the patient; and
the elongated introducer assembly also configured to puncture the biological feature of the patient and form a puncture site through the biological feature; and
the elongated introducer assembly also configured to dilate the puncture site; and
the elongated introducer assembly also configured to receive the auxiliary device for facilitation of movement of the auxiliary device toward the puncture site.

2. An apparatus for use with an auxiliary device and a biological feature of a patient, the apparatus comprising:
an elongated introducer assembly having a longitudinal axis extending therethrough, and including a distal portion configured to be selectively movable proximate to the biological feature of the patient; and
the distal portion including a puncture-treatment assembly being configured to puncture the biological feature of the patient and form a puncture site through the biological feature in response to selective movement of the distal portion toward the biological feature; and
the distal portion configured to dilate the puncture site in response to further selective movement of the distal portion through the puncture site after the puncture site is formed by the puncture-treatment assembly; and
the elongated introducer assembly and the distal portion defining an elongated introducer lumen configured to receive the auxiliary device for facilitation of movement of the auxiliary device through the puncture site extending through the biological feature.

3. The apparatus of claim 2, wherein:
the puncture-treatment assembly is configured to be selectively advanced toward a puncturing-tapered arrangement in preparation for formation of the puncture site.

4. The apparatus of claim 3, wherein:
the puncture-treatment assembly is also configured to be selectively retracted in preparation for dilation of the puncture site.

5. The apparatus of claim 2, wherein:
the puncture-treatment assembly is configured to be selectively collapsible or foldable toward a puncturing-tapered arrangement in preparation for formation of the puncture site.

6. The apparatus of claim 5, wherein:
the puncture-treatment assembly is also configured to be selectively expanded in preparation for dilation of the puncture site.

7. The apparatus of claim 2, wherein:
the puncture-treatment assembly includes:
movable spines configured to be positioned in a spaced-apart circumferential relationship around an outer circumference of the distal portion.

8. The apparatus of claim 2, wherein:
the puncture-treatment assembly is configured to be circumferentially axially extendable, axially along the longitudinal axis, from the distal portion.

9. The apparatus of claim 2, wherein:
the puncture-treatment assembly is also configured to selectively extend into, and retract outwardly from, the distal portion of the elongated introducer assembly.

10. The apparatus of claim 2, wherein:
the distal portion, of the elongated introducer assembly, is configured to selectively deflect the distal portion in response to movement of the puncture-treatment assembly.

11. The apparatus of claim 2, wherein:
the puncture-treatment assembly is configured to selectively deflect the distal portion of the elongated introducer assembly.

12. The apparatus of claim 2, wherein:
the puncture-treatment assembly is configured to conduct energy for puncturing the biological feature for formation of the puncture site.

13. The apparatus of claim 2, wherein:
the distal portion forms a dilation-tapered arrangement, in which a frontal section of the puncture-treatment assembly is covered, at least in part, by the distal portion.

14. The apparatus of claim 13, wherein:
the dilation-tapered arrangement is configured to dilate, at least in part, the puncture site formed by the puncture-treatment assembly.

15. The apparatus of claim 14, wherein:
the dilation-tapered arrangement presents a sloped dilation surface, and is sloped relative to the longitudinal axis.

16. The apparatus of claim 15, wherein:
the dilation-tapered arrangement is configured to dilate a size of the puncture site, up to the size of an outer diameter of the elongated introducer assembly.

17. The apparatus of claim 2, wherein:
the elongated introducer assembly includes a pull device; and
the pull device is configured to be reciprocally movable along the longitudinal axis of the elongated introducer assembly.

18. The apparatus of claim 17, wherein:
the puncture-treatment assembly is fixedly attached to the pull device.

19. The apparatus of claim 2, wherein:
the distal portion presents a tapered guide configured to guide movement of the puncture-treatment assembly.

20. A method of positioning an auxiliary device to a location being proximate to a biological feature of a patient, the method comprising:
selectively moving an elongated introducer assembly, having a longitudinal axis extending therethrough, and including a distal portion, proximate to the biological feature of the patient, in which the elongated introducer assembly and the distal portion define an elongated introducer lumen; and
puncturing the biological feature of the patient and forming a puncture site through the biological feature with a puncture-treatment assembly of the distal portion by selectively moving the distal portion toward the biological feature; and
dilating the puncture site with the distal portion by further selective movement of the distal portion through the puncture site after the puncture site is formed by the puncture-treatment assembly; and
facilitating movement of the auxiliary device along the elongated introducer lumen and toward the puncture site.
